(19) United States
(12) Patent Application Publication
(10) Pub. No.: US 2026/0124151 A1
(43) Pub. Date: May 7, 2026

(54) OGA-OVEREXPRESSED EXOSOME SYSTEM, PREPARATION METHOD THEREFOR AND USE THEREOF

(71) Applicant: NANJING DRUM TOWER HOSPITAL, Nanjing (CN)

(72) Inventors: Jinglin WANG, Nanjing (CN); Haozhen REN, Nanjing (CN)

(73) Assignee: NANJING DRUM TOWER HOSPITAL, Nanjing (CN)

(21) Appl. No.: 19/002,801

(22) Filed: Dec. 27, 2024

(30) Foreign Application Priority Data
Nov. 7, 2024 (CN) 202411584403.5

(51) Int. Cl. A61K 9/50; A61K 35/28; A61P 1/16; C12N 5/0775; C12N 15/86

(52) U.S. Cl. CPC A61K 9/5068; A61K 35/28; A61P 1/16; C12N 5/0662; C12N 15/86; C12N 2500/40; C12N 2510/00; C12N 2740/15043

(57) ABSTRACT

A bionic OGA-overexpressed exosome system for treating fatty liver-related liver cancer based on glucose metabolism and a preparation method therefor are provided. The O-GlcNAcase (OGA) sequence is transfected into mesenchymal stem cells by lentivector transfection, and exosomes are then obtained by ultracentrifugation for the treatment of fatty liver-related liver cancer. The exosome biomimetic system of the invention have the ability to target migration, low immunogenicity and improved ability to transport bioactive substances, normalize the interrupted glucose metabolism, reduce the endoplasmic reticulum stress and inhibit the epithelial-mesenchymal transition signal transduction. In vitro and in vivo experiments proved that MSCOGA-EXOs can effectively target cancer cells in vitro, reduce the abnormal O-GlcNAc modifications, and inhibit the malignant behavior of tumor. After reaching fatty liver-related hepatocellular carcinoma in vivo, the MSCOGA-EXOs can reduce the O-GlcNAc modification level and restore the damaged metabolism, thus inhibiting the progression of cancer.

### CROSS REFERENCE TO THE RELATED APPLICATIONS

This application is based upon and claims priority to Chinese Patent Application No. 2024115844035, filed on Nov. 7, 2024, the entire contents of which are incorporated herein by reference.

### TECHNICAL FIELD

The present invention belongs to the field of biomedical materials and, in particular, relates to a bionic OGA-overexpressed exosome system for treating fatty liver-related liver cancer based on glucose metabolism and a preparation method therefor and use thereof.

### BACKGROUND

As the prevalence of obesity increases, the proportion of fatty liver-related liver cancer in hepatocellular carcinoma also increase. Surgical resection is the main clinical treatment for this type of tumor, and drug therapy is also used as an intervention strategy to prevent tumor recurrence and improve patient survival. Drug therapies often target multiple signaling pathways to promote the death of tumor cells. Specifically, intervention through metabolic pathways plays an important role in inducing tumor cell apoptosis and inhibiting tumor metastasis. In these pathways, the regulation of glucose metabolism, especially in the context of fatty liver and overnutrition, is essential to inhibit tumor growth. As a result, many anti-tumor treatments targeting glucose metabolism have been developed. However, despite significant advances, mechanism of glucose metabolism in fatty liver-related liver cancer is still poorly understood. Therefore, the development of effective treatment strategies based on glucose metabolism is still worth looking forward to.

By sequencing the clinical excision samples of fatty liver-related liver cancer, it was found that the O-GlcNAcase (OGA) in these tumor tissues was significantly reduced. Previous studies have shown that a sharp increase in O-linked N-acetylglucosamine (O-GlcNAc) modification plays a key role in abnormal tumor glucose metabolism. However, the underlying mechanism of O-GlcNAc modification remains unexplored to great extent, leading to unsatisfactory results when targeting O-GlcNAc with drugs alone. It was found that OGA, a key enzyme that dynamically and temporarily regulates O-GlcNAc, was down-regulated in fatty liver-related liver cancer. By increasing the OGA level, abnormal O-GlcNAc modifications can be reduced, thereby restoring disrupted energy metabolism and alleviating endoplasmic reticulum (ER) stress. It is believed that the regulation of OGA simultaneously regulates the function of metabolic reprogramming and energy-related organelles, providing a promising pathway for targeted therapy of liver cancers.

Compared with conventional mesenchymal stem cells, exosomes have the ability to target migration, low immunogenicity and efficient transport of bioactive substances, so they are adaptable and controllable in therapeutic applications. Therefore, to confirm this hypothesis, an OGA-overexpressed mesenchymal stem cell (MSC^{OGA}) exosome system (MSC^{OGA}-EXOs) is developed for the treatment of fatty liver-related liver cancer.

### SUMMARY

Objective of invention: In order to solve the above technical problems, the invention provides a bionic OGA-overexpressed exosome system (MSC^{OGA}-EXOs) for the treatment of fatty liver-related liver cancer based on glucose metabolism. The exosome system is prepared by lentivector transfection and ultracentrifugation. The method is simple, versatile and convenient for large-scale production.

The technical solution: An OGA-overexpressed exosome system (MSC^{OGA}-EXOs) of the invention is obtained by lentivector transfection for transfecting an O-GlcNAcase (OGA) sequence into human mesenchymal stem cells (MSCs), followed by ultracentrifugation, wherein the exosomes are spherical in shape, with an average diameter of 120 nm-125 nm.

The specific steps are as follows:
1) preparation of human MSCs;
2) preparation of OGA-overexpressed human mesenchymal stem cells (MSC^{OGA}): diluting OGA-overexpressed lentivector particles with a medium by lentivector transfection, adding the MSCs into the medium for co-incubation in an incubator, and adding purinomycin to select infected cells expressing resistance genes until no cells die; and
3) preparation of OGA-overexpressed exosomes (MSC^{OGA}-EXOs): culturing the human MSCs in a medium lacking exosomes, and performing primary centrifugation and secondary centrifugation on the medium to obtain the exosomes system.

Preferably, the titer of the OGA-overexpressed lentivector particles in step 2) is 10⁹ copies/mL.

Preferably, the medium in step 2) is Dulbecco's modified Eagle's medium/Ham's F12 (DMEM/F12) medium and a volume ratio of the OGA-overexpressed lentivector particles to the DMEM/F12 medium is 1:1000.

Preferably, the concentration of the human MSCs in the medium is 10⁶ cells/mL.

Preferably, the concentration of the purinomycin added to the medium in step 2 is 2 μg/mL.

Preferably, the primary centrifugation in step 3 is: at room temperature, centrifuging the medium at 300×g for 10 min, then at 2000×g for 10 min, and then at 10,000×g for 30 min, and resuspending pellet particles in phosphate buffer saline (PBS).

Preferably, the secondary centrifugation in step 3 is: centrifuging the pellet particles obtained from the primary centrifugation at 100000×g, 4° C. for 70 min, removing supernatant and obtaining pellets which are exosomes.

The invention further provides an application of the OGA-overexpressed exosome system in the preparation of a drug for fatty liver-related liver cancer.

Preferably, the drug is a fatty liver-related liver cancer drug based on glucose metabolism.

### Beneficial effects

(1) The prevent invention designs MSC^{OGA}-EXOs for fatty liver-related liver cancer, which have superior ability to target migration.

(2) The MSC^{OGA}-EXOs for fatty liver-related liver cancer according to the present invention are prepared by lentivector transfection and ultracentrifugation, and the method is simple, is operated conveniently, and has strong repeatability, low technical requirements, strong versatility, high flexibility and easy for large-scale production.

(3) The MSC^{OGA}-EXOs for fatty liver-related liver cancer according to the present invention have the ability to target migration, low immunogenicity and improved ability to transport bioactive substances, normalize the interrupted glucose metabolism, reduce the endoplasmic reticulum stress (ERS) and inhibit the epithelial-mesenchymal transition (EMT) signal transduction. In vitro and in vivo experiments proved that MSC^{OGA}-EXOs can effectively target cancer cells in vitro, reduce the abnormal O-GlcNAc modifications, and inhibit the malignant behavior of tumor. After reaching fatty liver-related hepatocellular carcinoma in vivo, the MSC^{OGA}-EXOs can reduce the O-GlcNAc modification level and restore the damaged metabolism, thus inhibiting the progression of cancer.

### BRIEF DESCRIPTION OF THE DRAWINGS

FIGS. 1A-1H show expression profile analysis of O-GlcNAc related markers in patients with fatty liver-related liver cancers: (FIG. 1A) schematic of RNA sequencing, immuno-histochemistry, and Western blotting using clinical samples from patients with fatty liver-related liver cancers; (FIG. 1B) comparison of 3-year disease-free survival (DFS) between patients with fatty liver-related liver cancers and patients with other liver cancers; (FIG. 1C) comparison of 3-year DFS in liver cancer patients with and without metabolic syndrome; (FIG. 1D) Cox proportional hazard regression analysis of baseline DFS data for patients; (FIG. 1E) RNA sequencing for differentially expressed genes in carcinoma and para-carcinoma tissues of patients with fatty liver-related liver cancers; (FIG. 1F) expression levels of OGA and OGT in carcinoma (group C) tissues and para-carcinoma (group P) tissues of patients with fatty liver-related liver cancers, detected by Western blotting; (FIG. 1G)-(FIG. 1H) immuno-histochemical staining of OGA and OGT expression in carcinoma and para-carcinoma tissues of patients with fatty liver-related liver cancers, with a scale of 100 μm.

FIGS. 2A-2G show preparation and characterization of MSC^{OGA}-EXOS: (FIG. 2A) transfection of MSCs by lentivector system and secretion of exosomes; (FIG. 2B) morphologies of Vector and OGA-infected MSCs, shown by green fluorescence of EGFP, at a scale of 50 μm; (FIG. 2C)-(FIG. 2D) positive and negative surface markers of MSCs infection with OGA, detected by flow cytometry; (FIG. 2E) morphology of exosomes obtained by TEM images at a scale of 50 μm; (FIG. 2F) size distribution of MSC^{OGA}-EXOs by nanoparticle tracking analyzer (NTA); (FIG. 2G) exosomes identified by Western blotting.

FIGS. 3A-3J show the intake and function of MSC^{OGA}-EXOs in liver tumor cells: (FIG. 3A)-(FIG. 3D) the intake of MSC^{VEC}-EXOs and MSC^{OGA}-EXOs by tumor cells and primary hepatocytes at a scale of 10 μm; (FIG. 3E) expression of OGA and OGT in tumor cells (group T) and primary hepatocytes (group P) before and after intake of MSC^{OGA}-EXOs by Western blotting; (FIG. 3F)-(FIG. 3G) the migration of untreated tumor cells, MSC^{VEC}-EXOs-treated tumor cells, and MSC^{OGA}-EXOs-treated tumor cells at 0, 12, and 24 hours, at a scale of 100 μm; (FIG. 3H) untreated tumor cells (Con) and the migration of MSC^{VEC}-EXOs (Vec) and MSC^{OGA}-EXOs of different concentrations to tumor cells at 0, 12, and 24 hours at a scale of 100 μm; (FIG. 3I) ERS levels of untreated tumor cells (Con), MSC^{Vec}-EXOs-treated tumor cells (Vec) and tumor cells treated with different concentrations of MSC^{OGA}-EXOs, detected by Western blotting; (FIG. 3J) EMT levels of untreated tumor cells (Con), MSC^{VEC}-EXOs-treated tumor cells (Vec), and tumor cells treated with different concentrations of MSC^{OGA}-EXOs.

FIGS. 4A-4F show in vivo evaluation of MSC^{OGA}-EXOs in mice with orthotopic fatty liver-related liver cancer: (FIG. 4A) construction of STAM model mice simulating fatty liver-related liver cancer and exosome treatment; (FIG. 4B) In vivo imaging of mice in each group after injection: NC: normal mice injected with PBS; NC-Vec: normal mice injected with MSC^{Vec}-EXOs; NC-OGA: normal mice injected with MSC^{OGA}-EXOs; STAM: STAM mice injected with PBS; STAM-Vec: STAM mice injected with MSC^{Vec}-EXOs; STAM-OGA: STAM mice injected with MSC^{OGA}-EXOs; (FIG. 4C)-(FIG. 4E) body weight, liver weight and liver/body ratio of mice in six groups; (FIG. 4F) HE staining of carcinoma tissues and para-carcinoma tissues in STAM, STAM-Vec, and STAM-OGA mice at a scale of 400 μm.

FIGS. 5A-5G show in vivo evaluation of the therapeutic effect of MSC^{OGA}-EXOs on mice with fatty liver-related liver cancers: (FIG. 5A)-(FIG. 5C) expression levels of ALT, AST and AFP in mice in six groups; (FIG. 5D)-(FIG. 5E) expression levels of OGA and OGT in carcinoma tissues and para-carcinoma tissues, at a scale of 50 μm; (FIG. 5F)-(FIG. 5G) expression levels of ERS and EMT in mice in six groups, detected by Western blotting.

### DETAILED DESCRIPTION OF THE EMBODIMENTS

In order to deepen the understanding of the invention, the invention is further elaborated below in conjunction with the embodiments and the accompanying drawings. The embodiments are only used to explain the invention and do not constitute a limitation of the scope of the invention.

Human mesenchymal stem cells (MSCs) used in the examples below were purchased from American Type Culture Collection (ATCC).

### Example 1 Preparation Method of MSC^{OGA}-EXOs

### (1) Preparation of Human Mesenchymal Stem Cell (MSC):

Cells were cultured in a 75T culture bottle. The cells were cultured in a constant-temperature cell incubator at 5% CO₂, 37° C. Passage was performed when the cells grew to cover 80-90% of the bottom area of the culture bottle. First, the old medium in the petri dish was removed, the cells were washed twice with 3 ml of neutral phosphate buffer saline (PBS); then, 3 ml of 0.25% trypsin was added for digestion in the incubator at 37° C. for 2-3 min, and then 3 ml of the above medium was added to terminate digestion. The cell suspension was pipetted into a 15 ml centrifuge tube for centrifugation at 1000 rpm for 5 min. Then, the cells were resuspended with 3 ml of the above medium and passed into a new culture bottle at a ratio of 1:3.

### (2) Preparation of OGA Lentivector Transfection

MSC^{OGA} was prepared by stable transduction of lentivector carrying human OGA gene (10⁹ copies/mL). After the OGA lentivector particles were diluted with DMEM/F12 medium at a volume ratio of 1:1000, the MSC prepared in step 1 was added into the cell culture medium, and the concentration of the MSCs in the medium was 10⁶ cells/mL After incubation for 24 h, the medium containing lentiviral particles was replaced with fresh medium and incubated for another 48 h. Infected cells expressing resistance genes were selected by adding purinomycin, which was given a concentration of 2 μg/mL in the medium until no cells died. The expression of OGA was detected by qPCR and Western blot. After transfection, the purity of the cells was detected by flow cytometry and positive and negative markers were detected.

### (3) Preparation of OGA-Overexpressed Exosome System (MSC^{OGA}-EXOs)

To extract exosomes, MSCs were cultured in a medium lacking exosomes. The medium underwent a series of centrifugation steps: at room temperature, the medium was centrifuged at 300×g for 10 min, then at 2000×g for 10 min, then at 10000×g for 30 min; the supernatant was collected and pellets were resuspended in PBS; the pellets in PBS were centrifuged again at 4° C., 100000×g for 70 min, and after removal of the supernatant, the resulting pellets were exosomes.

### Example 2 Expression Profile Analysis Of O-GlcNAc-Related Markers in Patients with Fatty Liver-Related Liver Cancers

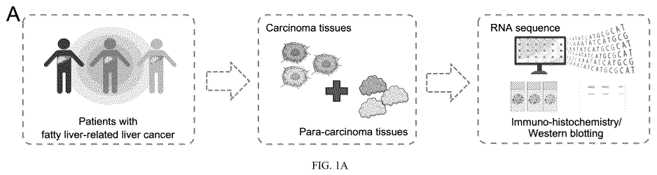

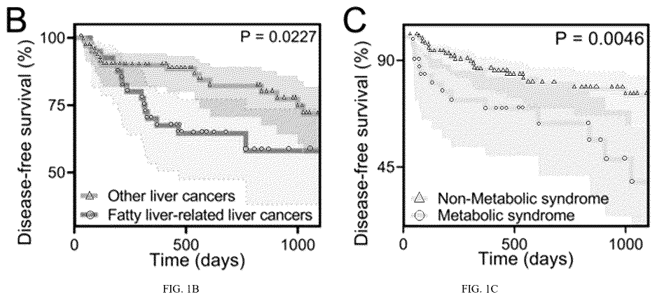

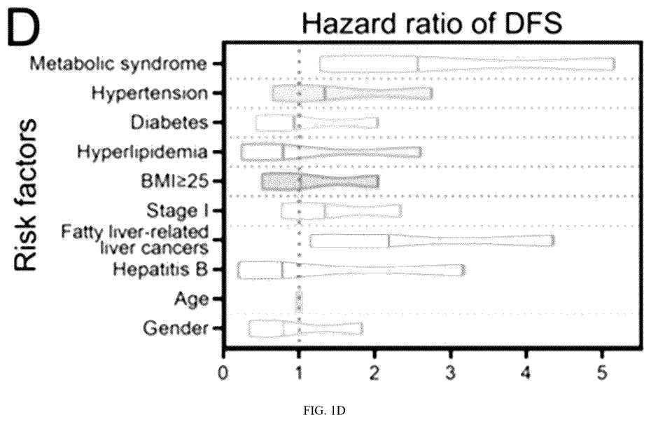

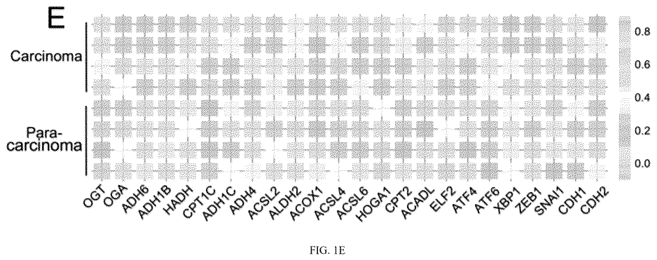

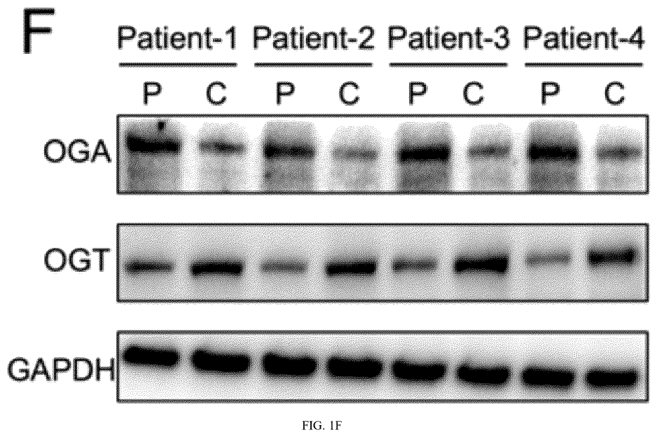

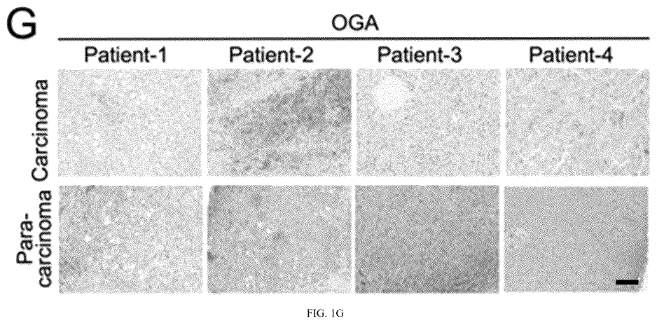

To confirm the effect of overnutrition on the malignancy of fatty liver-related liver cancer, we conducted a retrospective analysis on the prognosis of patients undergoing surgery for fatty liver-related liver cancer (FIG. 1A). On the one hand, we compared 3-year disease-free survival (DFS) and overall survival in patients with fatty liver-related liver cancers with those with other liver cancers. The results showed that the 3-year DFS of patients with liver cancer combined with fatty liver was significantly lower than that of patients with other liver cancers (FIG. 1B). On the other hand, we studied 3-year DFS and overall survival in patients with and without metabolic syndrome. The results showed that 3-year DFS of patients with metabolic syndrome was significantly lower than that of patients without metabolic syndrome (FIG. 1C). To further figure out the factors affecting 3-year DFS and overall survival, we performed a Cox proportional hazard regression analysis of baseline patient data. Analysis showed that fatty liver (univariate analysis, hazard ratio [HR], 2.188; 95% confidence interval [CI], 1.097-4.365) and metabolic syndrome (univariate analysis, HR, 2.564; 95% CI, 1.275-5.157) were independent risk factors for 3-year DFS in patients with liver cancers (FIG. 1D). These findings suggest that metabolic reprogramming, one of the top ten features of malignant tumors, plays an integral role in the development of fatty liver-related liver cancers.

In different metabolic reprogramming, glucose metabolism disorder is considered to be a key link in the development of fatty liver and overnutrition and liver cancer. However, the understanding of the role of O-GlcNAc modification in fatty liver-related liver cancers is still limited. To elucidate the O-GlcNAc modification mechanism and its potential targets associated with fatty liver-related liver cancers, we analyzed samples from patients undergoing clinical surgery using RNA sequencing, immuno-histochemistry, and western blotting. Through RNA sequencing of carcinoma and para-carcinoma tissues of patients with fatty liver-related liver cancers, we found that compared with para-carcinoma tissues, genes responsible for glycosylation, especially genes related to O-GlcNAc transferase (OGT), were up-regulated in carcinoma tissues (FIG. 1E). Conversely, the expression of genes involved in deglycosylation, mainly OGA, was found to be reduced. Immuno-histochemistry and Western blotting results also showed that the expression level of OGT was high in fatty liver-related carcinoma, while the expression level of OGA was lower (FIGS. 1F-1H). These observations suggest that reduced expression of OGA in hepatocellular carcinoma related to fatty liver disease may be associated with poor patient prognosis. Therefore, given the critical role of OGA in the removal of OGlcNAc modifications, reduced expression of OGA in these hepatocellular carcinoma tissues may lead to poor prognosis.

### Example 3 Preparation and Characterization of MSC^{OGA}-EXOs

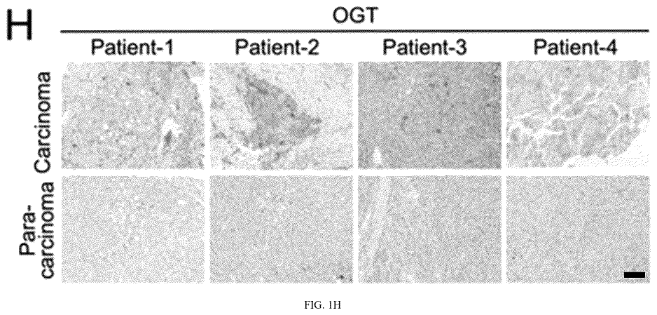

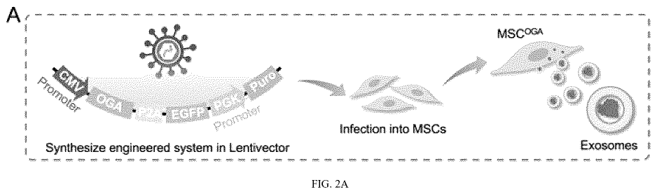

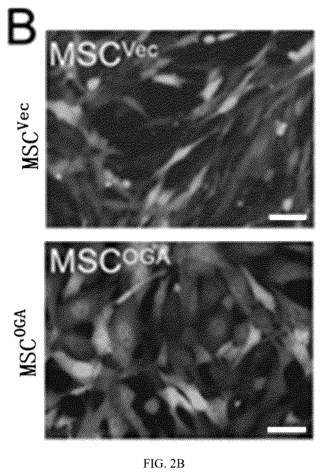

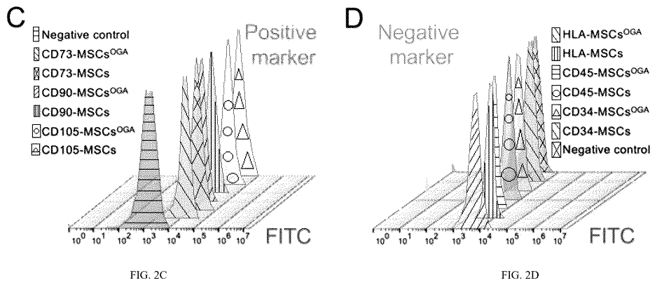

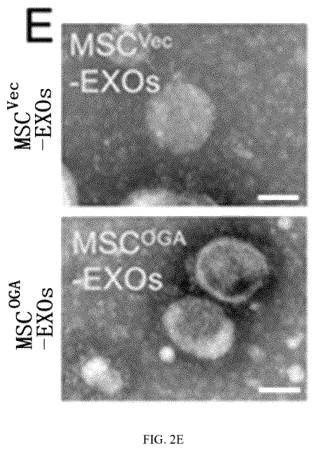

We developed an MSC^{OGA} exosome system with high expression of OGA (MSC^{OGA}-EXOs) for the treatment of fatty liver-related liver cancers (FIG. 2A). In view of this, MSCs were infected with a lentivector carrying the OGA gene and selected with purinomycin. Under the microscope, it was observed that the infected MSCs maintained a spindle shape (FIG. 2B). To verify that lentivector transduction does not induce differentiation of MSCs, the expression of positive and negative surface markers specific to MSCs was detected. Flow cytometry analysis confirmed that OGA-modified MSCs continued to express CD73, CD90, and CD105, but not CD34, CD45, and HLA-DR (FIGS. 2C-2D). In order to obtain the desirable exosomes, supernatant centrifugation was performed to further isolate exosomes from MSCs. Transmission electron microscopy (TEM) revealed that MSC^{OGA}-EXOs are spherical in shape with an average diameter of about 123 nm (FIGS. 2E-2F). In addition, exosomes were identified using membrane proteins such as D63, CD81, and the membrane-associated protein TSG101 (FIG. 2G). These data indicate that OGA-overexpressed MSC-derived exosomes (MSC^{OGA}-EXOs) had been obtained successfully.

### Example 4 Evaluation on Intake and Function of MSC^{OGA}-EXOs in Liver Tumor Cells

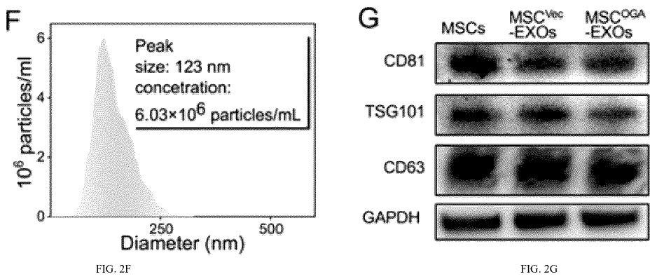

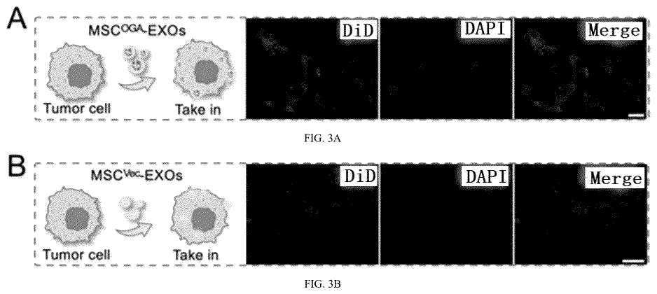

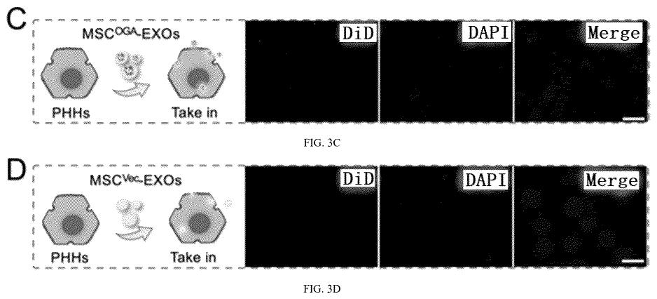

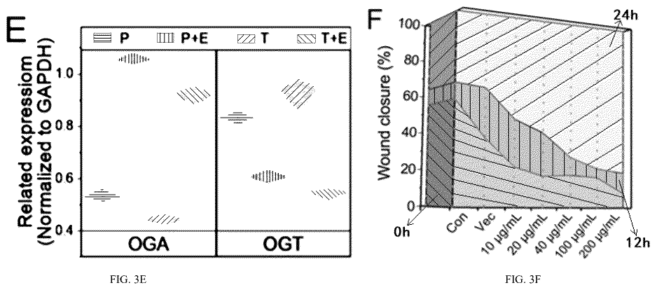

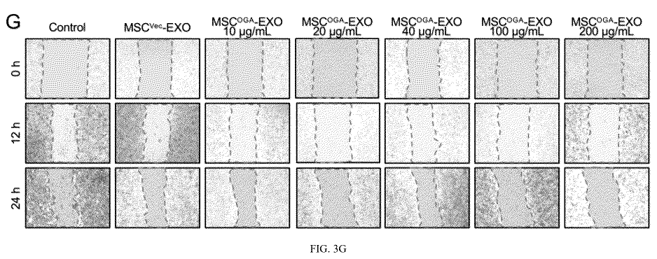

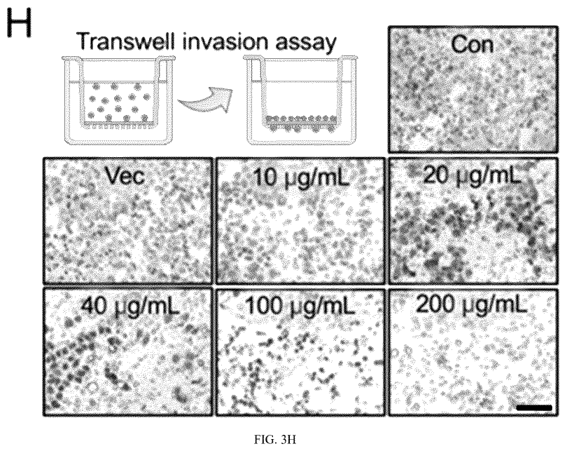

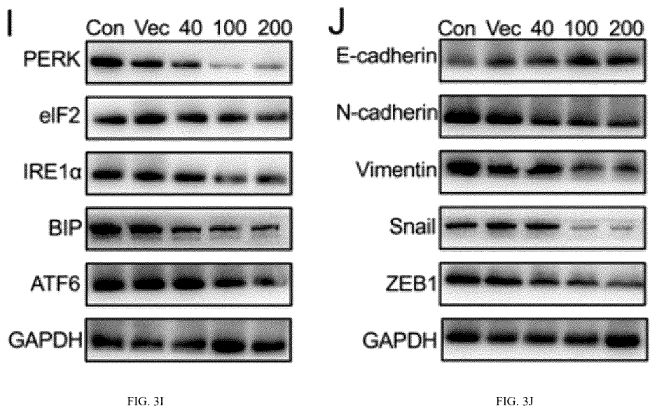

After production of MSC^{OGA}-EXOs, their functions were evaluated with liver tumor cells and primary hepatocytes to examine their uptake efficiency. MSC^{OGA}-EXOs were labeled with 1,1′-dioctadecyl-3,3,3′,3′-tetramethylindodicarbocyanine,4-chlorobenzenesulfonate (DiD) and co-incubated with the cells for 24 h, and the results were observed using confocal laser scanning microscopy. The resulting images showed red fluorescence in the cytoplasm, confirming the cellular internalization of exosomes (FIGS. 3A-3D). Interestingly, a curious phenomenon was observed that the intake of DiD by tumor cells was higher than that of hepatocytes, which indicates that tumor cells have a more active phagocytosis. This phenomenon is conducive to the effective intake of the prepared exosomes by the tumor cells, so that they can play the expected role.

To verify the effect of exosome intake on the overall O-GlcNAc modification level of tumor cells, the expression of OGA and OGT proteins in these cells were detected by western blotting. The results showed that compared with the primary hepatocyte group (group P), the O-GlcNAc modification level of tumor cells (group T) was increased, OGT production was enhanced, and OGA expression was decreased. This effect was reversed after the introduction of the MSC^{OGA}-EXOs. In addition, tumor cells with intake of MSC^{OGA}-EXOs (T+E group) showed significantly higher OGA expression and reduced OGT production compared to primary hepatocytes with intake of MSC^{OGA}-EXOs (P+E group) (FIG. 3E). To determine the effect of OGA on hepatocyte progression, migration and invasion experiments were performed to assess how exosome treatment-induced changes in OGA expression affect the migration and invasion characteristics of hepatocellular carcinoma cells. These experimental data showed that the migration and invasion potential of hepatocellular carcinoma cells treated with OGA was significantly reduced (FIGS. 3F-3H).

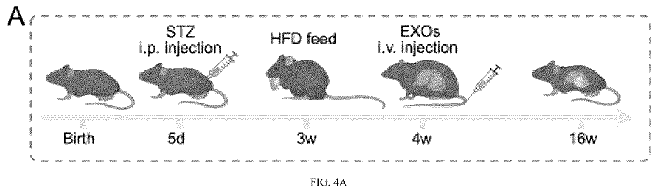

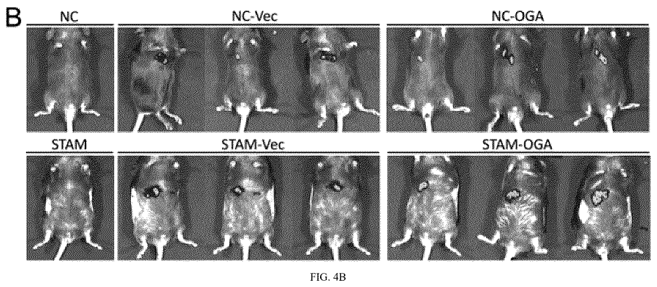

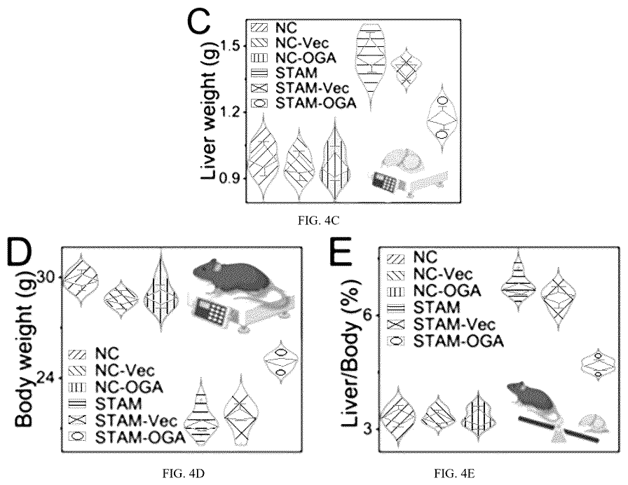

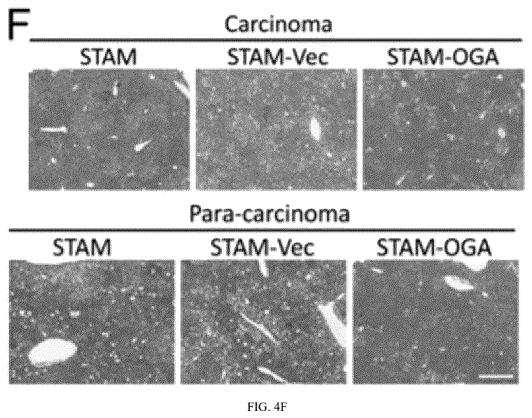

In order to figure out the effects of MSC^{OGA}-EXOs intake on ER, ER stress was assessed by Western blotting. The results showed that overexpression of OGA could inhibit ER stress, and with the increase of exosome concentration, ER stress could be further inhibited (FIG. 3I). In addition, we also explored the mechanism of OGA regulating the migration and invasion of hepatocellular carcinoma cells. By western blotting, it was confirmed that overexpression of OGA could up-regulate the expression levels of EMT-related proteins, such as ZEB1, Snail, N-cadherin and Vimentin, indicating that treatment with MSC^{OGA}-EXOs can inhibit the occurrence of EMT in hepatocellular carcinoma cells (FIG. 3J). Therefore, from all cellular levels in FIGS. 4A-4F, it was found that tumor cells could internalize exosomes, overexpress OGA, and with OGA intake, tumor cells show reduced ability to migrate and invade. In addition, these results suggest that our MSCOGA-EXOs can effectively mitigate the malignant behavior of tumor cells and is expected to play a role in tumor therapy.

### Example 5 In Vivo Evaluation of MSC^{OGA}-EXOs in Mice with Orthotopic Fatty Liver-Related Liver Cancer

To investigate the actual role of OGA gene in fatty liver-related liver cancer in vivo, MSCOGA-EXOs test was researched using Stelic animal models (STAM) and normal diet models (FIG. 4A). For the STAM model, male mice were intraperitoneally injected with 200 g of streptozotocin (STZ) within 5 days of birth and then given a 60% high-fat diet (HFD) at 3 weeks of age. Mice with normal diet and STAM model mice were divided into three groups and injected at the 4th week with phosphate buffer saline (NC, STAM), MSC^{Vec}-EXOs (NC-Vec, STAM-vec) and MSC^{OGA}-EXOs (NC-OGA, STAM-OGA) respectively, and then sampled when the mice reached 16 weeks of age. To confirm in vivo targeting ability, bioluminescence imaging was used to track exosomes entering the body. From the imaging results, it was observed that most exosomes were localized in the liver, indicating their targeting ability and subsequent potential to exert anti-tumor effects in the liver (FIG. 4B). By measuring mouse livers and body weight, we further analyzed the effect of MSC^{OGA}-EXOs on tumorigenesis after targeting liver (FIGS. 4C-4E). Although the mice with fatty liver weighed more than control mice, they actually weighed less at 16 weeks due to tumor progression. In addition, compared with control mice, STAM mice also had a higher liver weight due to tumor load, resulting in a larger liver/body ratio. In addition, the body weight of all mice in the STAM-OGA group was higher than that of mice in the STAM-Vec group, and the liver/body ratio of all mice was significantly lower than that of mice in the STAM-Vec group. It is worth mentioning that the liver of STAM mice had obvious carcinogenic effect, with single or multiple tumors. In contrast, the tumor size of the STAM-OGA group was significantly reduced as compared with the other groups (FIG. 4F). These data suggest that our liver-targeted MSCOGA-EXOs can significantly inhibit the occurrence and progression of fatty liver-related liver cancers.

### Example 6 In Vivo Evaluation of the Therapeutic Effect Of MSC^{OGA}-EXOs on Fatty Liver-Related Liver Cancers in Mice

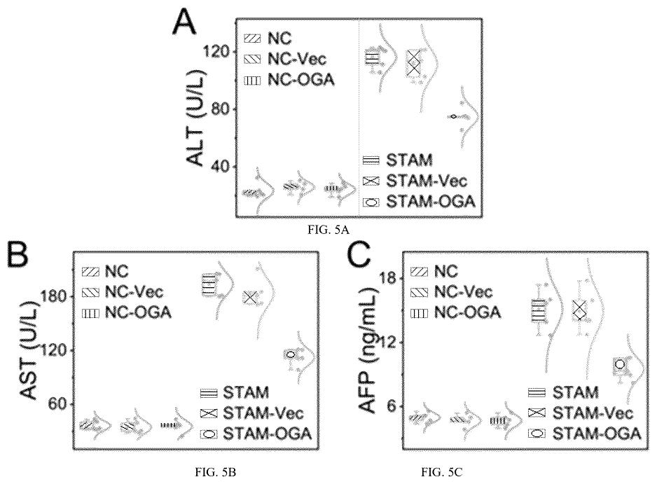

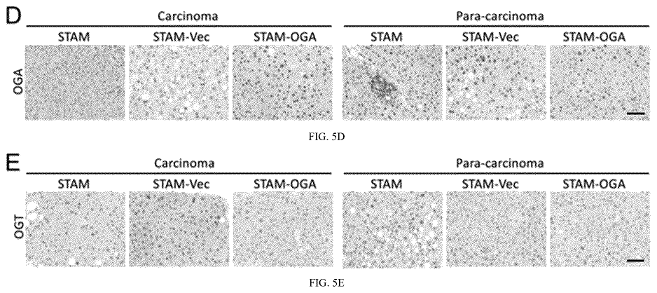

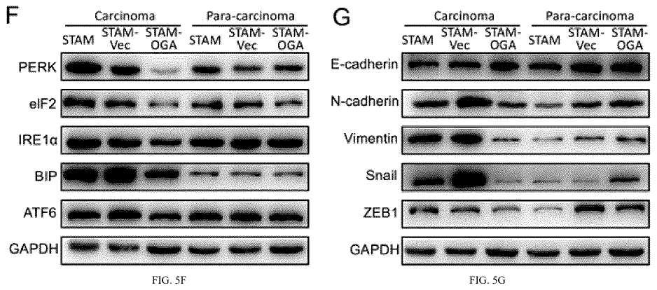

To further evaluate the effect of MSC^{OGA}-EXOs on tumor progression, function indicators and alpha-fetoprotein (AFP) levels of mice with liver cancer were also evaluated. Compared with mice having normal diet, the levels of alanine aminotransferase (ALT), aspartate aminotransferase (AST) and alpha-fetoprotein (AFP) in serum of mice in three STAM groups were increased. However, after treatment with MSC^{OGA}-EXOs, the ALT, AST, and AFP levels were significantly lower in the STAM-OGA group than in the STAM and STAM-Vec groups (FIGS. 5A-5C). This demonstrates the improvement of liver function and the effectiveness of anti-tumor therapy in the STAM-OGA group. These positive results prompted us to further investigate the expression levels of OGA and OGT in these mice. The results also showed that OGA expression was effectively enhanced and OGT level decreased in the STAM-OGA group (FIGS. 5D-5E), indicating that MSC^{OGA}-EXOs can effectively regulate the recovery of dysregulated O-GlcNAc modification, thereby controlling tumor progression.

To clarify the inhibitory mechanism of MSC^{OGA}-EXOs, we also detected in vivo ER stress (ERS) and EMT levels, both of which are indicators of tumor-related malignancy. Our results in the STAM-OGA group showed a significant reduction in ER stress levels, indicating that MSCOGA-EXOs can significantly inhibit ER stress, thereby inhibiting tumor progression (FIG. 5F). In addition, we studied the proteins associated with EMT. The results showed that the cell phenotype changed and the expression of E-cadherin decreased, leading to the decrease of cell adhesion and the acquisition of invasive and migratory characteristics. It was also found that loss of E-cadherin expression was the most significant feature of EMT in mice with fatty liver-related liver cancer, and exosome therapy prevented EMT progression (FIG. 5G). These results suggest that our exosomes can be used as a viable strategy for EMT treatment of fatty liver-related liver cancer in mice, paving the way for new therapeutic approaches for cancer treatment.

The above are only preferred embodiments of the invention and are not used to limit the invention, any modification, equivalent replacement, improvement, etc. made within the spirit and principle of the invention shall fall within the scope of the invention.

What is claimed is:

1. An O-GlcNAcase (OGA)-overexpressed exosome system, wherein the OGA-overexpressed exosome system is obtained by a lentivector transfection for transfecting an OGA sequence into human mesenchymal stem cells (MSCs), followed by an ultracentrifugation, wherein exosomes are spherical in shape, with an average diameter of 120 nm-125 nm.

2. A preparation method for the OGA-overexpressed exosome system according to claim 1, comprising the following steps:
1) a preparation of the human MSCs;
2) a preparation of OGA-overexpressed human MSCs (MSC^{OGA}): diluting OGA-overexpressed lentivector particles with a medium by the lentivector transfection, adding the human MSCs into the medium for a co-incubation in an incubator, and adding purinomycin to select infected cells expressing resistance genes until no cells die; and
3) a preparation of OGA-overexpressed exosomes: culturing the human MSCs in a medium lacking the exosomes, and performing a primary centrifugation and a secondary centrifugation on the medium lacking the exosomes to obtain the OGA-overexpressed exosome system.

3. The preparation method according to claim 2, wherein a titer of the OGA-overexpressed lentivector particles in the step 2) is 10⁹ copies/mL.

4. The preparation method according to claim 2, wherein the medium in the step 2) is a Dulbecco's modified Eagle's medium/Ham's F12 (DMEM/F12) medium, and a volume ratio of the OGA-overexpressed lentivector particles to the DMEM/F12 medium is 1:1000.

5. The preparation method according to claim 2, wherein a concentration of the human MSCs in the step 2) in the medium is 10⁶ cells/mL.

6. The preparation method according to claim 2, wherein a concentration of the purinomycin added to the medium in the step 2) is 2 μg/mL.

7. The preparation method according to claim 2, wherein the primary centrifugation in the step 3) is: at a room temperature, centrifuging the medium lacking the exosomes at 300×g for 10 min, then at 2000×g for 10 min, and then at 10,000×g for 30 min, and resuspending pellet particles in phosphate buffer saline (PBS).

8. The preparation method according to claim 2, wherein the secondary centrifugation in the step 3) is: centrifuging pellet particles obtained from the primary centrifugation at 100000×g, 4° C. for 70 min, and removing a supernatant and obtaining pellets, wherein the pellets are the exosomes.
